(19) United States
(12) Patent Application Publication
(10) Pub. No.: US 2015/0147806 A1
(43) Pub. Date: May 28, 2015

(54) METHOD FOR PREPARING TRANSFERRABLE NANOSCALE TRANSFERRABLE MEMBRANE AND USE THEREOF

(71) Applicant: SNU R&DB Foundation, Seoul (KR)

(72) Inventors: Kookheon CHAR, Seoul (KR); Jwa-Min NAM, Seoul (KR); Yeongseon JANG, Seoul (KR); Hyojin LEE, Seoul (KR)

(73) Assignee: SNU R&DB FOUNDATION, Seoul (KR)

(21) Appl. No.: 14/549,186

(22) Filed: Nov. 20, 2014

Related U.S. Application Data
(60) Provisional application No. 61/907,604, filed on Nov. 22, 2013.

(51) Int. Cl. C12M 1/12; B29C 47/00; C12N 5/09

(52) U.S. Cl. CPC C12M 25/02; C12N 5/0693; B29C 47/0021; C12N 2502/1352; B29K 2001/12
USPC 435/347; 435/297.1; 435/373; 435/396; 435/401; 264/51

(57) ABSTRACT

Disclosed is a method for preparing a transferable membrane having a nanometer scale dimension in thickness and pore size by non-solvent vapor-induced phase separation process, comprising spin-casting a polymer solution in a closed humid chamber and controlling the relative humidity (RH) of the chamber using at least one supersaturated salts solution whereby the density of the pores are controlled. Also provided is a TNT membrane prepared by the present method and its use. The present membrane can be advantageously used as co-culture platform facilitating versatile and controllable cell co-culture assays and further allowing the quantitative analysis of paracrine communications between cells for example between cancer cells and different types of stromal cells by providing an in vivo-like environment, which can offer more in-vivo-like results to identify key signaling molecules for therapeutic targets of a disease.

### CROSS-REFERENCE TO RELATED APPLICATIONS

This application claims the benefit of priority to U.S. Provisional Application No. 61/907,604 filed Nov. 22, 2013 in United States Patent and Trademark Office, disclosure of which is incorporated herein by reference.

### BACKGROUND OF THE INVENTION

1. Field of the Invention

The present disclosure generally relates to a method for preparing a nanoscale membrane and a cell culture technology using the membrane made by the same which allows assays based on a cell-cell communication in an in vivo like environment.

2. Description of the Related Art

Cells within a tissue interact with neighboring cells or extracellular matrices (ECM) by biochemical and mechanical cues to maintain functions of tissues and homeostasis Thus for the effective mucous delivery, a higher dose or a repeated administration of the drug is required which causes inconvenience to the patients. In many biological systems, cells communicate with each other generally through direct contact (i.e., gap junction, tight junction) or trade of soluble factors (i.e., paracrine and endocrine signaling through cytokines, chemokines, and growth factors).

For example, cancer development has been considered as the result of evolving crosstalk between tumor cells and surrounding various types of stromal cells including fibroblasts, myoblasts, immune cells, mesenchymal stem cells. Tumor cells constantly modulate their stromal environment by producing a range of growth factors, which activate the stromal cells in a paracrine manner as well as disrupt normal tissue homeostasis, leading to the secretion of additional growth factors and proteases. The activated stromal cells also promote tumor metastasis by secreting growth factors and matrix metalloproteinases (MMPs) that degrade and remodel the ECM.

Therefore, the development of highly efficient cell co-culture platforms is needed to systematically investigate the paracrine communications between cancer cells and stromal cells to fully understand and control the tumor cell behavior.

Many previous studies on the paracrine signaling were mainly conducted by using membrane-separated cell co-culture platforms which mediate the indirect contact between different cell types (Kim, S. et al. A novel culture technique for human embryonic stem cells using porous membranes. Stem Cells 25, 2601-2609 (2007)).

However, the deficiency in cytokine-mediated cell-cell interactions in the membrane-based co-culture systems as described above was reported as a major reason for the loss of in vivo-like functionality of cells of interest (Karnoub, A. E. et al. Mesenchymal stem cells within tumour stroma promote breast cancer metastasis. Nature 449, 557-U554, (2007)). This limited cell-cell interaction is mainly derived from the separation of cell population by a membrane with low pore density and micrometer-scaled thickness. Moreover, millimeter-scaled distance between two different cell lines induces dissolution of cytokines by external fluid and enzymes as well as optically non-transparent membrane impose difficulties in imaging and analyzing specific cells of interest.

According to other conventional techniques, various kinds of cells were directly co-cultured on tissue culture substrates to study the cell-cell interactions in vitro (Wallace, C. S. & Truskey, G. A. Direct-contact co-culture between smooth muscle and endothelial cells inhibits TNF-alpha-mediated endothelial cell activation. Am J Physiol-Heart C 299, H338-H346, (2010)). Although this approach offers in vivo-like cell communication environment, there exist still numerous technical difficulties to be resolved such as cross-contamination of cells by xenogenic reaction, isolation of a specific cell line, flexibility in ways of interesting different cell lines, and difficulties in the separate analysis on each cell lines (Cenni, E., Perut, F. & Baldini, N. In vitro models for the evaluation of angiogenic potential in bone engineering. Acta Pharmacol Sin 32, 21-30, (2011)). Further the most serious problem in the direct contact system is the evaluation of the relative synthesis of soluble factors because the total cytokine expression content of each cell type cannot be distinguished.

U.S. Pat. No. 8,599,939 published on Jul. 5, 2012 discloses a method of cell cultures and device for implanting it in which a cell culture vessel having at least one first zone and at least one second zone, wherein the first zone is a transfer zone for a culture medium which essentially contains no cells and the second zone is a cell culture zone. The invention further includes methods utilizing the cell culture vessel.

Thus, there exist needs for the development of cell co-culture system or platform, which allows a more in-depth cell-cell interaction studies which plays a major role in many biological phenomenon such as cancer development and metastasis and the like.

### SUMMARY OF THE INVENTION

In one aspect, the present disclosure provides a method for preparing a freestanding, transferable membrane having a nanometer scale dimension in thickness and pore size by non-solvent vapor-induced phase separation process, comprising spin-casting a polymer solution in a closed humid chamber and controlling the relative humidity (RH) of the chamber using at least one supersaturated salts solution whereby the density of the pores are controlled.

In the present disclosure the thickness of the membrane prepared is controlled by a spinning rate, a solution concentration, and a solubility parameter between the polymer and the solvent used. These conditions are intimately connected to one another to decide the membrane thickness. In one embodiment, the solution concentration below 4 wt % and the spinning rate over 3000 rpm are needed to get a thin membrane with a thickness below 500 nm.

The polymer that may be used for the present methods are determined in consideration of type of nonsolvent or solvent used. The type of polymer may affect solute adsorption, membrane hydrophilicity, and the thermal and chemical stability of the membrane. In nonsolvent induced phase separation, the choice of polymer limits the solvents and nonsolvents that can be used in the phase inversion process. In addition, the solvent also plays an important role along with polymer concentration during membrane formation. Various polymers meeting such conditions may be employed for the present methods and there exists large numbers of compatible solvent-nonsolvent pairs, each with their own specific thermodynamic behavior and miscibility. In one embodiment, the polymer that may be employed for the present method includes but is not limited to cellulose acetate, cellulose acetate, polysulfone, polyethersulfone, polyarylonitrile, cellulosics, poly(vinylidene fluoride), poly(tetrafluoroethylene), polyimide or polyamide.

In the present methods, relative humidity is controlled to obtain a target pore size of interest in the membrane. Salts are used to control the relative humidity, and the pore size is determined by RH, in which case, water is a nonsolvent. For example, to control the relative humidity, different kinds of supersaturated salt solution are used because the degree of hydration depends on the salt types. In one embodiment, LiCl, CaCl₂, MgCl₂, KCO₃, NaBr, NaCl, or KCl may be used to control the relative humidity to 11, 29, 33, 43, 57, 75, or 85% RH at room temperature or 30° C., respectively. In other embodiment, at least one supersaturated salt that is used for the present method is CaCl₂ and KCl and the relative humidity is increased gradually or in a stepwise manner from 25 to 85% wherein the CaCl₂ is used when the RH is controlled in the range of 25-45% and KCl is used when the RH is controlled in the range of 55-85%. In one embodiment, the relative humidity is increased in a stepwise manner from 25 to 85% with 35, 45, 55, 65, 75% in between, In one embodiment, the relative humidity is controlled to 25, 35, 45, 55, 65, 75 and 85%.

In one embodiment of the present method the relative humidity is controlled at 30° C. or RT.

In other aspect, the present disclosure provides a cell culture platform comprising: at least one membrane prepared by the present method having a plurality of pores in nanoscale extending between opposite sides thereof and a thickness of about 500 nm or less; and a first and a second chamber separated by the membrane.

In one embodiment, the first and second chambers contained in the cell culture platform are able to communicate with each other through the plurality of pores in nanoscale. In other embodiment, the first and second chambers contain a cell culture medium, the first chamber contains a first cell type, and the second chamber contains a second cell type. In other embodiment, the first cell type and the second cell type are identical or different, or at least one of the first cell type and the second cell type is a mixture of at least two different cell types.

In still other embodiment the present platform further comprises at least one intermediate chamber positioned between the first chamber and the second chamber, the intermediate chamber being separated from each of the first and second chambers by at least one nanoscale membrane.

In one embodiment, the number of intermediate chambers comprised in is 2 to 10.

In still other embodiment, the first, second and intermediate chambers contain a cell culture medium, and at least one of the chambers contain a same or a different cell type or a mixture of at least two different cell types.

In still other embodiment, the density of the pores in the nanoscale membrane is 10⁵ to 10⁶ pores per cm².

In still other embodiment, the average diameter of the pore is 50-100 nm.

In other aspect, the present disclosure provides a method for co-culturing two or more types of cells, comprising: providing at least one membrane prepared by the present method as described herein, wherein when the membranes comprised are two or more, the membranes are stacked in layers from top to bottom or from right to left; seeding cells on the membranes wherein the cells are of the same or different type or a mixture of at least two different types of cells; and culturing the cells whereby the cells are able to communicate with each other through the pores of the membrane.

In one embodiment of method, the communication is by a direct contact through gap junctions or tight junction or by the exchange of soluble factors through paracrine or endocrine signaling.

In other embodiment of the method, the cells are the cells are originated from an animal, plant, bacteria, fungus, yeast or algae.

In still other embodiment of the method, the cells seeded are cancer cells or stromal cells, and each of the cancer cells and stromal cells are seed on a different membrane.

In still other embodiment of the method, the positions of the membranes are changed relative to each other, or at least one of the membrane is removed and replaced with a fresh membrane, and the fresh membrane contains cells that are different from the cells contained in the existing membranes.

In other aspect, the present disclosure provides a cell co-culture kit which comprises: the membrane according to the present disclosure, one or more cell culture media; one or more cell lines; and instructions for co-culturing the one or more cell lines using the kit.

The present membrane produced by the above disclosed method provides a freestanding Transparent, Nanoporous, Transferrable (TNT) membrane. The present membrane can be advantageously used as co-culture platform facilitating versatile and controllable cell co-culture assays and further allowing the quantitative analysis of paracrine communications between cells for example between cancer cells and different types of stromal cells by providing an in vivo-like environment, which could offer more in-vivo-like results to identify key signaling molecules therapeutic targets of a disease.

Also the present membrane or the platform can provide a handle to observe and analyze the specific cell types of interest when different cell types are co-cultured simultaneously. The present TNT membrane which can be co-stacked can be advantageously used for the studies for various types of cell assays and tissue engineering applications. For example, the differentiation of stem cells and cell-cell communications in nerve system can be tuned by changing the density and size of the membrane nanopores and the types and co-culture sequences of surrounding cells in a highly controlled and analyzable manner.

The foregoing summary is illustrative only and is not intended to be in any way limiting. Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1A is a representation of the process for preparing the nanoporous and freestanding CA membranes by non-solvent (water) vapor-induced phase separation (VIPS) in a closed spin-coating chamber with controlled relative humidity (RH) using the devices depicted on the left part.

FIG. 1B are AFM images of the TNT (Transparent, Nanoporous, Transferrable membranes) (a) on the air side (b) on the substrate side and the air side AFM images of TNT membranes were also taken (c) in air as well as (d) in contact with water.

FIG. 2A is characterization of TNT membranes prepared according to the present method having transparency, nanopores and transferability of ultrathin TNT membranes in aqueous environment.

FIG. 2B is a schematic representation of co-culture of MDA-MB-231 and hMSC using TNT membranes for paracrine signaling assay.

FIG. 2C is AFM images of TNT membranes prepared with 4 wt % acetone CA solution under various RH conditions (45, 65, and 85%). The number and size of pores in a TNT membrane was controlled by changing RH and the Inset scale bar is 500 nm,

FIG. 2D shows the effect of pore size on the RANTES expression triggered by the cytokine-mediated communications between MDA-MB-231 and hMSC.

FIG. 2E is a SEM image of a microporous 10 μm-thick-CA membrane prepared by non-solvent liquid induced phase separation (LIPS) (PET membrane).

FIG. 2F is the result showing the effect of membrane thickness on the RANTES expression.

FIG. 3A to FIG. 3E show TNT membrane-based cell co-stacking assays for cancer cell-stromal cell paracrine communication or signaling.

FIG. 3A is a schematic representation of the co-culture of MDA-MB-231 and three different types of stromal cells with TNT membrane co-stacking cell culture strategy.

FIG. 3B is the results of cytokine assay using the luminescence-based method which was obtained from cell culture media after the co-culture of MDA-MB-231 (M) with three different stromal cell lines [hMSC (H), NIH-3T3 (N) and C2C12 (C), named as MH, MN and MC, respectively for 2 days and the data are representative of multiple independent experiments and are expressed as means±standard deviation. (*; P<0.05, ***; P<0.01).

FIG. 3C is results of the migration assay of M using a transwell system and the optical density (O.D) at 560 nm represents the degree of migration and the inserted gel image is a result of gelatin zymography for comparison invasiveness of M and the band intensity indicates the activity of active MMP.

FIG. 3D is the fluorescence images of multi-stacked TNT membranes containing two different cell lines (N: Green and M: Red) obtained by confocal laser scanning microscopy (CLSM) and the DIC images of cells on TNT were observed by optical microscopy.

FIG. 3E is the result of the cytokine assay after the co-culture with three different cell lines multi-stacked with TNT membrane.

FIG. 4A and FIG. 4B show the results of Calcein AM detection of a gap junctional intercellular communication between MDA-MB-231 (A) and hMSC (B) indicating that there is no physical contact between MDA-MB-231 and hMSC since Calcein AM can only be transported through the gap junction in cellular membrane into live cells.

FIGS. 5A and 5D show protein diffusion through the TNT membrane.

FIG. 5A shows the effect of porous membrane thickness on RANTES expression, triggered by the paracrine signaling between MDA-MB-231 and hMSC.

FIG. 5B shows the effect of cell-cell distance on the RANTES secretion. FIG. 5C shows protein diffusion through the nanopores of the TNT membrane (insert: a schematic of the experimental setup for measuring the diffusion of EGF through the TNT membrane).

FIG. 5D shows the protein diffusion result as a function of the number of membrane stacking layers using epidermal growth factor (EGF) as a model protein.

FIG. 6A to 6B show TNT membrane-based sequential cell transfer and shuffling (TNT-CTS) assays.

FIG. 6A is a representative schematic diagram of cancer metastasis and metastasis-mimicking TNT-CTS assay. The TNT membrane containing M was cultured with stromal cells (H, N or C) and then transferred to another stromal cell-containing TNT layer.

FIG. 6B is the result of cytokine assay according to stromal cell type, co-cultured with M for 1 day. Pre-activated M with H (red) is transferred to N (yellow) or C (blue).

FIG. 6C is the result when pre-activated M with N (yellow) is transferred to H (red) or C (blue).

FIG. 6D is the result when pre-activated M with C (blue) is transferred to H (red) or N (yellow).

FIG. 7A to FIG. 7E each show platforms comprising the present membrane according to various embodiment of the present disclosure.

### DETAILED DESCRIPTION OF THE EMBODIMENTS

The present disclosure is further explained in more detail with reference to the following examples. These examples, however, should not be interpreted as limiting the scope of the present invention in any manner.

In one aspect, the present disclosure relates to a method for preparing a transferable membrane having a nanometer scale dimension in thickness and pore size by non-solvent vapor-induced phase separation process, comprising spin-casting a polymer solution in a closed humid chamber and controlling the relative humidity (RH) of the chamber using at least one supersaturated salts solution whereby the density of the pores are controlled.

The membrane prepared by the present method is a Transparent, Nanoporous, Transferrable (TNT) membrane and has a thickness of 500 nm or less and a plurality of pores in nanoscale extending between opposite sides thereof through which the cells cultured thereon can communicate to each other as described hereinafter. Also the present membrane is freestanding and thus does not require a substrate for support and is facilitated in stacking and destacking.

A general information for non-solvent vapor-induced phase separation process may be found in Guillen, Y. Pan, M. Li, and E. M. V. Hoek, Preparation and Characterization of Membranes Formed by Nonsolvent Induced Phase Separation: A Review, Ind. Eng. Chem. Res. 2011, 50, 3798-3817.

In the present methods, the thickness of the membrane is controlled by a spinning rate, a solution concentration, and/or a solubility parameter between a polymer and a solvent employed. These conditions are intimately related to one another to determine the membrane thickness. In one embodiment, the solution concentration below 4 wt % and the spinning rate over 3000 rpm are used to get a thin membrane with a thickness below 500 nm.

In accordance with the present method, the membrane may be prepared to a specification considering the particular purpose of interest of the present membrane. For example, membranes which allow a cell-cell communication may be obtained by controlling the thickness, the size of pores and/the pore density.

In one embodiment, the thickness of the membrane is about 500 nm or for example about 490 nm or less, or about 480 nm or less, or about 470 nm or less.

Also in the present methods, polymers that may be employed for the present methods are determined in consideration of type of nonsolvent or solvent used. The type of polymer used may affect solute adsorption, membrane hydrophilicity, and the thermal and chemical stability of the membrane. In nonsolvent induced phase separation, the choice of polymer limits the solvents and nonsolvents that can be used in the phase inversion process. In addition, the solvent also plays an important role along with polymer concentration during membrane formation. Various polymers meeting such conditions may be employed for the present methods and there exists large numbers of compatible solvent-nonsolvent pairs, each with their own specific thermodynamic behavior and miscibility. Such materials include biocompatible organic polymers having properties such as low electrostatic, high mechanical strength, and/or whose microstructural properties are easy to be controlled. In one embodiment, the polymer that may be employed for the present method includes but is not limited to cellulose acetate, polysulfone, polyethersulfone, polyarylonitrile, cellulosics, poly(vinylidene fluoride), poly(tetrafluoroethylene), polyimide or polyamide.

In the present methods, relative humidity is controlled to obtain a target pore size of interest in the membrane. Supersaturated salts are used to control the relative humidity, and the pore size is determined by RH, in which case, water is a nonsolvent. For example, to control the relative humidity, different kinds of supersaturated salt solution are used because the degree of hydration depends on the salt types. In one embodiment, LiCl, CaCl₂, MgCl₂, KCO₃, NaBr, NaCl, or KCl may be used to control the relative humidity to about 11, 29, 33, 43, 57, 75, or 85% RH at room temperature or 30° C., respectively. In other embodiment, at least one supersaturated salt that is used for the present method is CaCl₂ and KCl and the relative humidity is increased gradually or in a stepwise manner from about 25 to 85% wherein the CaCl₂ is used when the RH is controlled in the range of about 25-45% and KCl is used when the RH is controlled in the range of about 55-85%. In one embodiment, the relative humidity is increased in a stepwise manner from about 25 to 85% with 35, 45, 55, 65, 75% in between, In one embodiment, the relative humidity is controlled to 25, 35, 45, 55, 65, 75 and 85%.

In one embodiment, also as described in Example of the present disclosure, a non-solvent Vapor Induced Phase separation method (N-VIPS) was used using cellulose acetate and controlling relative humidity (RH) to obtain the porous structure of the membrane, in which the size of the pores is controlled by RH (i.e., water vapors) in a closed chamber packed with different types of supersaturated salt solutions.

In accordance with the present methods, the size of the pores of the membrane may be prepared to a specification according to the particular purpose of interest of the present membrane. In one embodiment, the size of the pore ranges from about 100 nm to 500 nm. The pore size of the membrane which may be used for culturing cells may be various depending on the types of cell-cell interactions analyzed. For example when paracrine signals involving the transfer of biochemicals among cells are required, the pore size of less than 400 nm is preferred, and when cell-cell interactions through direct contact between cells are required, the pore size of at least 400 nm is preferred.

The density of the pores of the membrane may be prepared in a specification according to the particular purpose of interest of the present device. In one embodiment, membranes with a high density, for example such as about 10⁶ to 10¹²/cm² particularly 10⁵ to 10⁶ pores per cm² for cell culture may be employed.

In other aspect, the present disclosure relates to a cell culture platform comprising: at least one membrane prepared by the present method having a plurality of pores in nanoscale extending between opposite sides thereof and a thickness of about 500 nm or less; and a first and a second chamber separated by the membrane.

In one embodiment, the first and second chambers contained in the cell culture platform are able to communicate with each other through the plurality of pores in nanoscale.

The term “communication” or “communicate” refers to a direct or indirect interaction of cells, the former being interactions including a direct exchange of materials through gap junction or tight junction, the latter being interactions through a paracrine or endocrine signal transduction via exchange of soluble factors such as cytokines, chemokines and/or growth factors and the like secreted from cells.

The membrane which is employed for the present platform may be coated on one or both sides of the membrane or modified to have a certain material at the pores to improve or have additional functionality according to the particular purpose of interest. For example, in one embodiment, the membrane may be coated to improve attachment of the cells to the membrane using for example polydopamine, or Ku, S. H., et al., General functionalization route for cell adhesion on non-wetting surfaces. Biomaterials 31, 2535-2541 (2010) may be referred for further information. In other embodiment, the membrane may be modified with various biological materials such as receptors, antibodies, ligands or any low or high molecular weight materials which can interact with materials present on the surface of cells.

In other embodiment, the first and second chambers contain a cell culture medium, the first chamber contains a first cell type, and the second chamber contains a second cell type. In other embodiment, the first cell type and the second cell type are identical or different, or at least one of the first cell type and the second cell type is a mixture of at least two different cell types.

In still other embodiment the present platform further comprises at least one intermediate chamber, i,e, 3^{rd}, 4^{th} or 5^{th} chambers positioned between the first chamber and the second chamber, the intermediate chamber being separated from each of the first and second chambers by at least one nanoscale membranes. In one embodiment, the number of intermediate chambers comprised in is 2 to 10.

In still other embodiment, the present device comprises three chambers, in which a third chamber is positioned between a first and a second chamber, wherein each of the chamber is separated by the membrane.

The present platform may comprise cell culture media and a variety of cell lines of the same or different type in alone or in combination to study cell-cell interactions and/or communications. The cells included in each chamber may be present as attached on to the membrane or as buoyant. The cells which may be included are not limited to a particular cell type and comprises cells originated from animals, plants, bacteria, fungi, yeasts, and algae. In one embodiment, cells originated from various tumor or stromal cells are used.

In one embodiment, the membrane comprised in the present platform has a density of the pores in the range of 10⁵ to 10⁶ pores per cm² and an average diameter of the pore in the range of 50-100 nm.

The platform comprising the present nanoscale membrane has a housing. The housing which may be used for the present platform is made of materials which are suitable for cell culture, such as for example ceramics, polycarbonate, vinyl, polyvinylchloride, polydimethylsiloxane, acryl, polypropylene, polyethylene, polyethylsulfone or metals.

The housing employed for the present platform may be configured to have a structure that is suitable for a particular use of the present device. For example, the housing may take a form of a test tube suitable for cell culture or centrifuge, or a form of a cell culture plate. Or the housing may be connected to microfluidics and may further comprise a cover.

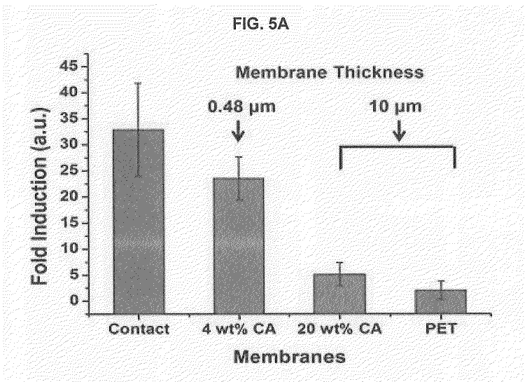

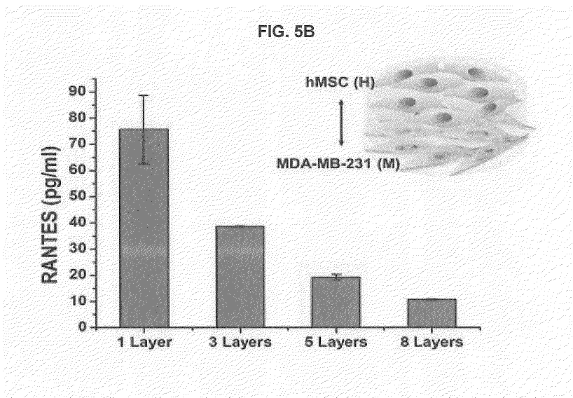

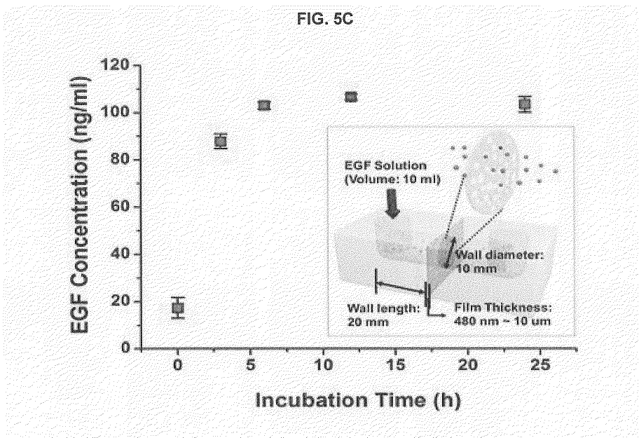

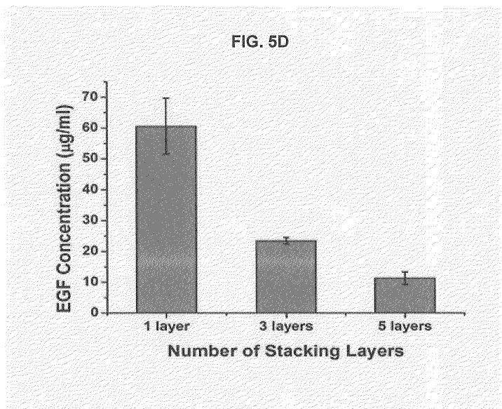

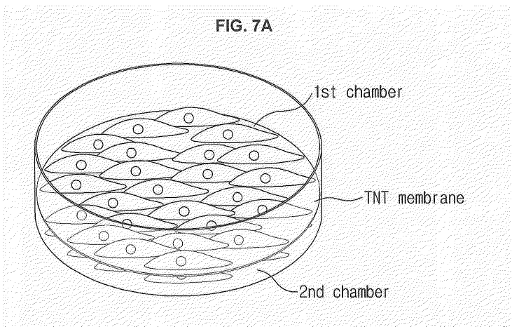

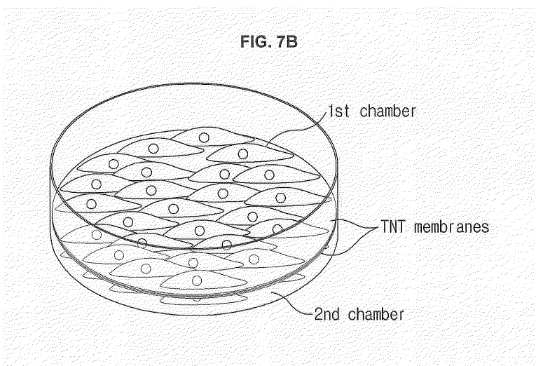

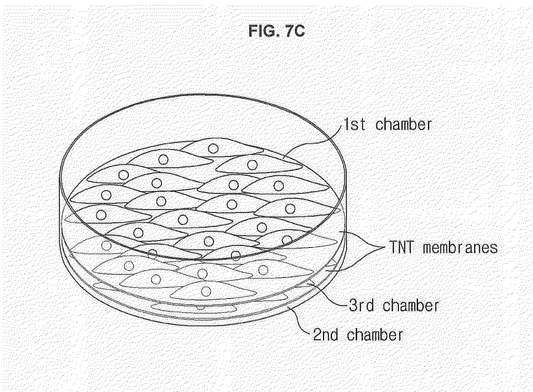

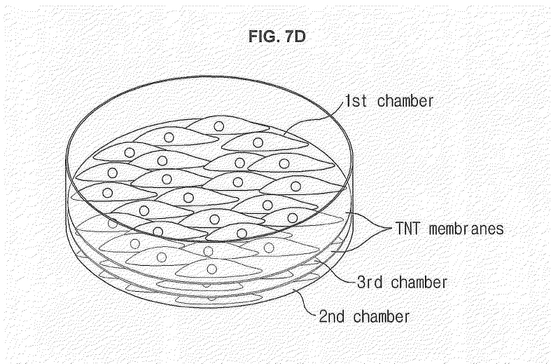

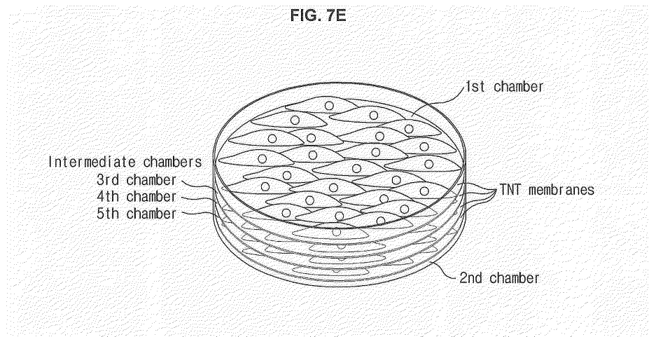

The present platform may take various forms. In one embodiment, referring to FIGS. 7A and 7B and 5C (inserted picture), the present platform comprises a first chamber and a second chamber and at least one nanoscale membrane located therebetween. Typical platform employing such structures includes test tubes or plates in which a first and a second chamber separated by at least one membrane. In other embodiment, the present device may further comprise at least one intermediate chamber for example a third chamber between a first and a second chamber as depicted in FIGS. 7C to 7E. In one embodiment, the present platform may comprise at least two chambers, and each chamber is arranged in a layer from right to left or from top to bottom.

Referring to FIGS. 7A to 7E, the present platform may further comprise media for cell culture and the cells may be present on one or both sides of the membrane, and the cells present on one or both sides of the membrane may be of identical or different type. The cells which are co-cultured in the present platform reside on the membrane as a single layer and interact with other only through the membrane.

All or part of the chambers included in the present device may comprise a medium for cell culture, and all or part of the chamber may include cells or cell lines.

As described hereinbefore, various cell lines may be employed for the present device to analyze interactions between cells and interactions between media and cells. For example, the cells contained in each chamber may be of identical or different type of cells or a mixture of at least two different cell types. The cells which may be employed for the present device comprise animal cells, plant cells, bacteria, fungi, yeast or algae.

Further the cells which may be employed for the present device include primary cells from tissues or organs or established cells. Primary cells include for example cells derived from organs such as liver, kidney, lungs, stomach, spleen, nerve, muscle, and bone in addition to bone marrow cells, cancer stem cells, mesenchymal stem cells, immune cells for example T cells, antigen presenting cells, dendritic cells, epithelial cells for example keratinocytes, blood vessel cells for example blood vessel endothelial cells, blood vessel muscle cells, nerve cells without being limited thereto. Established cells include for example HeLa cells, HeLa cells, FL cells, KB cells, HepG2 cells, WI-88 cells, MA104 cells, BSC-1 cells, Vero cells, CV-1 cells, BHK-21 cells, L cells, CHL cells, BAE cells, BRL cells, PAE cells, MDA-MB-231 cells.

In one embodiment, cancer cells or stromal cells interacting with cancer cells for example, fibroblast, myoblast and mesenchymal stem cells may be employed for the present device, but the cells are not limited thereto.

The medium which may be employed for the present device may various depending on particular cells cultured. For example general media include but are not limited to Fisher's medium (Gibco), Basal Media Eagle (BME), Dulbecco's Modified Eagle Media (DMEM), Iscoves's Modified Dulbecco's Media, Minimum Essential Media (MEM), McCoy's 5A Media, and RPMI; or media specialized for a specific cell include MyeloCuht™ (Stem Cell Technologies) and Opti-Cell™ (ICN Biomedicals) or serum free media include StemSpan SFEM™ (StemCell Technologies), StemPro 34 SFM (Life Technologies), and Marrow-Gro (Quality Biological Inc.). For example for endothelial cell culture, EBM2 (Bio Whittaker) may be used and for bone marrow cell culture, McCoy's 5A medium (Gibco) may be used. Such media may comprise as a supplement serum, antibiotics, amino acids and/or hormones.

In other aspects, the present disclosure further relates to a method for co-culturing at least two types of cells. The present method includes a step of providing at least one membrane prepared by the present method as described herein, wherein when the membranes comprised are two or more, the membranes are stacked in layers from top to bottom or from right to left; seeding cells on the membranes wherein the cells are of the same or different type or a mixture of at least two different types of cells; and culturing the cells whereby the cells are able to communicate with each other through the pores of the membrane.

In the present co-culturing method, the positions of the membranes are changed relative to each other, or at least one of the membranes is removed and replaced with a fresh membrane, and the fresh membrane contains cells that are different from the cells contained in the existing membranes.

In the methods according to the present disclosure, the communication includes a direct and indirect interaction, which is described hereinbefore. In one embodiment, the communication or interaction is an interaction through the membrane without physical contact between the cells. The present methods may be used for various cells types for cell-cell interaction or communications as described hereinbefore. In one embodiment, the cells which may be used for the present method include cancer cells or stromal cells or stromal cells which interact with cancer cells.

In other aspect, the present disclosure relates to a kit comprising the present platforms or membrane as described hereinbefore. The present kit may comprise at least one cell culture media; at least one cell lines; and/or an instruction for culturing the cells using the kit.

### EXAMPLES

### Example 1

### Preparation and Characterization of Porous Freestanding Polymer Thin Membrane

Cellulose acetate (CA) with average number of molecular weight (Mₙ) of 30,000 g/mol (39.8 wt % acetyl labeling extent) was obtained from Aldrich and used as received. CA was dissolved in acetone, with concentration of 4 wt %. To obtain porous structure of CA thin films, the spin-casting of CA solutions was performed with spinning rate 3000 rpm for 20 s using an automatic spin coater in a closed humid chamber with controlled relative humidity (RH) at 30 degree Celsius. Since water is a non-solvent for CA polymers, the porous structure in the CA thin films can be developed by non-solvent vapor induced phase separation (N-VIPS) and the number of pores was controlled by the RH (i.e., water vapor) in the closed chamber packed with different types of supersaturated salt solutions (CaCl₂ and KCl for RH 25-45% and 55-85%, respectively). Since the nanopores in the CA membranes was developed by the humidity-based VIPS, several discrete pores at 45% RH was gradually converted to well-defined pores with the maximum number pore density at 65% RH and finally leading to the bimodal pore morphology at RH 85%. The film thickness was maintained at around 500 nm independent of RH. The average value of the thickness of CA films was obtained from 6 different samples prepared under the same condition. The average value of the thickness of CA films prepared at 65% RH is 482.25 nm and the standard deviation for this set of data is 6.99 nm, implying that the porous thin films prepared in this study yield a highly uniform film thickness. The freestanding CA thin films were easily obtained by peeling off the films from the Si substrates in water or by immersing the CA-coated NaCl substrates into water for 10 min after sufficient drying of the samples. The porous 10-μm-thick CA films to check the film thickness effect were prepared by non-solvent liquid induced phase separation (N-LIPS): the 20 wt % CA solution dissolved in 2-ethyl-1,3-hexanediol was dropped onto a Si wafer constructed with 10-μm-sidewalls and casted by Dr. Blade method. Prepared CA films were immersed into water for 1 hr then dried in vacuum for 12 hrs, which makes the film easily detached from the substrate. The surface morphologies of CA films were characterized by AFM (dilnnova, Veeco Instruments Inc.) and FE-SEM (JSM-6701F, JEOL). The thicknesses of the CA thin films were obtained from step height measurement (AlphaStep IQ (Rev. Al-1), KLA Tencor). To enhance cell adhesion, the CA membranes were first coated by polydopamine in dopamine hydrochloride solution for up to 16 hrs. The dopamine hydrochloride (Sigma) was dissolved in 10 mM Tris buffer with concentration of 2 mg/ml, and the solution pH was adjusted at 8.5 with dilute NaOH solution. For basic principle and detailed characteristics of polydopamine coating refer to Ku, S. H., et al. General functionalization route for cell adhesion on non-wetting surfaces. Biomaterials 31, 2535-2541 (2010).

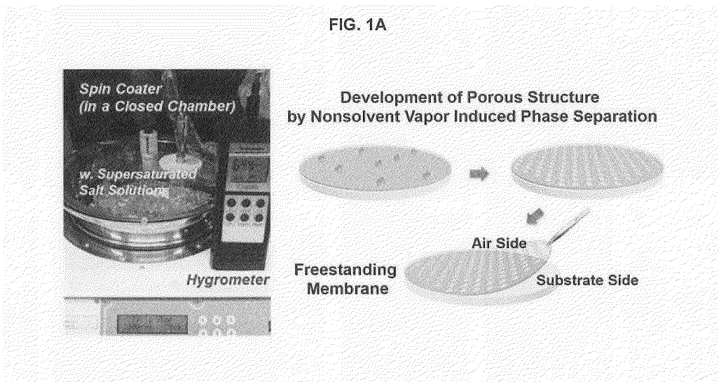

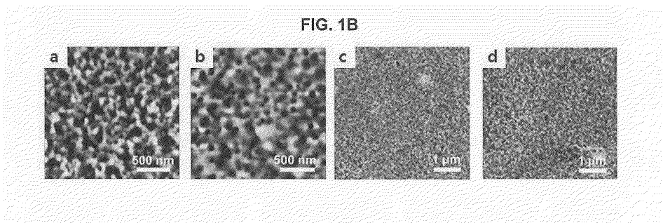

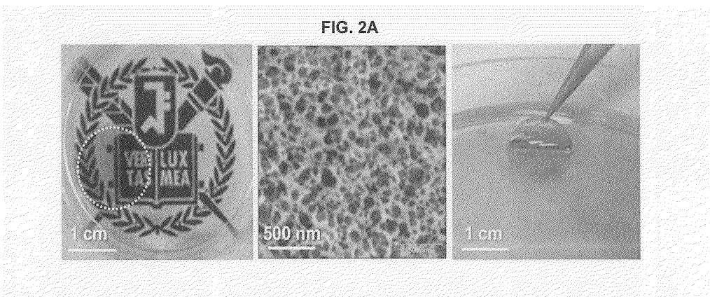

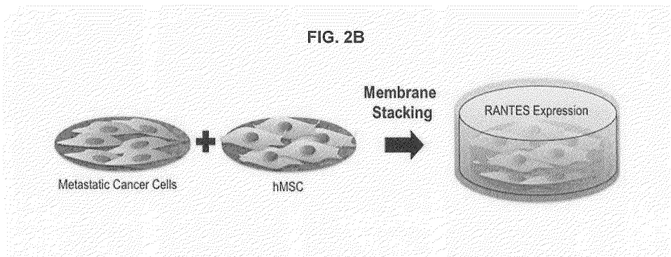

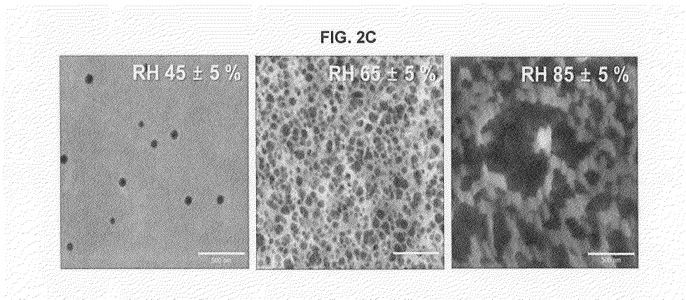

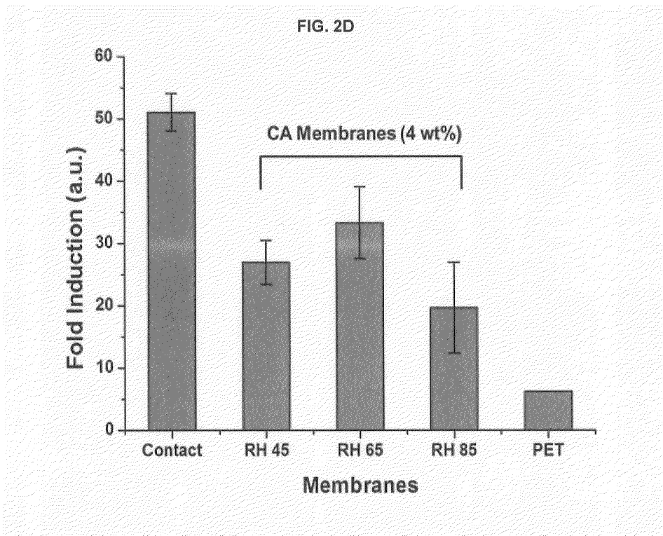

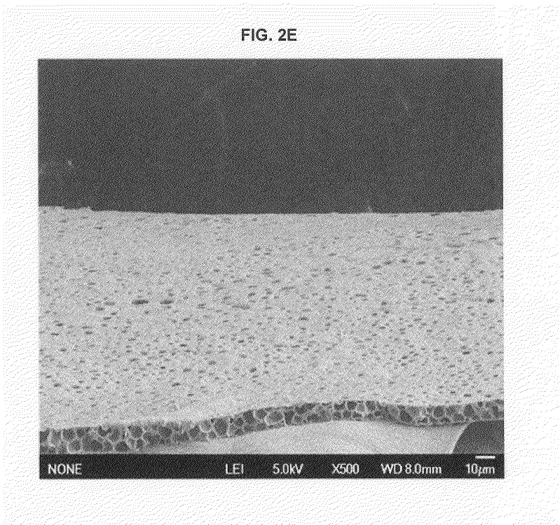

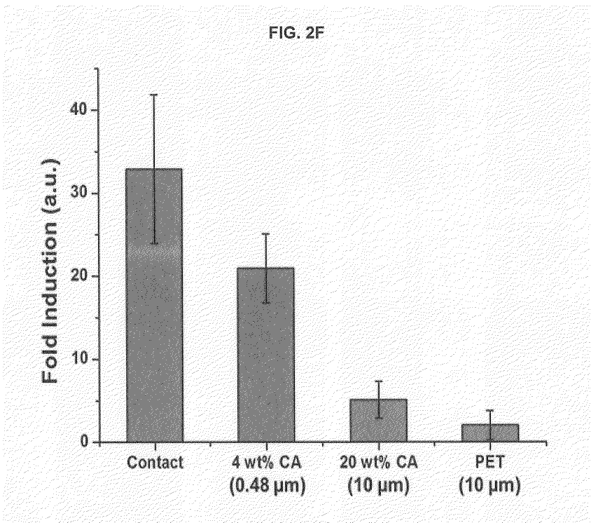

Results are shown in FIG. 1A to FIG. 2F. FIG. 1A is a representation of the process for preparing the nanoporous and freestanding CA membranes by non-solvent (water) vapor-induced phase separation (VIPS) in a closed spin-coating chamber with controlled relative humidity (RH) (right part) using the devices depicted on the left part. FIG. 1B are AFM images of the TNT membranes (a) on the air side (b) on the substrate side and the air side AFM images of TNT membranes were also taken (c) in air as well as (d) in contact with water. It was noted that the pore structure did not significantly alter even in aqueous environment.

As shown in FIG. 2A, the membrane according to the present disclosure is freestanding and shows the transparency, nanopores and transferability with an ultrathin thickness. Such transparency is due to the size of the pore (≦500 nm in diameter) and the nanometer scaled thickness (482±7 nm). Namely, these freestanding CA membranes with well-defined nanoporous structures have unique characteristics such as transparency and transferability in aqueous environment such as cell-culture media. Further as shown in FIG. 2C, according to AFM image of the TNT membrane prepared at different RH conditions (45, 65, and 85%), the pore size of the present TNT membranes can be regulated by controlling the relative humidity. Such membranes can be used for cell-cell paracrine communication, which is evident in FIG. 2D. FIG. 2D shows the effect of pore size on the RANTES expression triggered by the cytokine-mediated communications between MDA-MB-231 and hMSC, in which it was found that high density nanoporous (50-100 nm) membrane with a thickness of 475 nm prepared in RH 65% is the most efficient. Further in comparison the conventional PET (polyethylene terephthalate) membrane, high concentration of secreted protein RANTES was observed in the cell-cell communication experiment using the present membrane. Also FIG. 2E is a SEM image of a microporous 10 μm-thick-CA membrane prepared by non-solvent liquid induced phase separation (LIPS) which was used to assess the effect of thickness of the membrane on the expression of RANTES as a concentration of RANTES. As shown in FIG. 2F the communication between cells is strongly correlated with the cell-cell distance related to membrane thickness.

### Example 2

### Co-Culture of Cells Using the Present Membrane

Cells used in the present disclosure were cultured as follows. MDA-MB-231 (ATCC Num. HTB-26), NIH-3T3 (ATCC Num. CRL-1658), and C2C12 (ATCC Num. 1772) were purchased from American type culture collection and human mesenchymal stem cell (hMSC) was purchased from Merck Millipore (Part # SCC034, MA, USA). The MDA-MDA-231, metastatic breast carcinoma cell, was culture in 10% fetal bovine serum (FBS) contained RPMI media (Gibco, USA) with 100 units/ml penicillin-streptomycin (Gibco, USA) The NIH-3T3 and C2C12 were grown in DMEM (Gibco, USA) with 10% FBS and antibiotics. The hMSC were cultured in mesenchymal stem cell expansion medium (SCM015, Merck Millipore, USA). And we used the hMSC between 4^{th} and 8^{th} passages. All cell lines were incubated at 37° C., 5% CO₂.

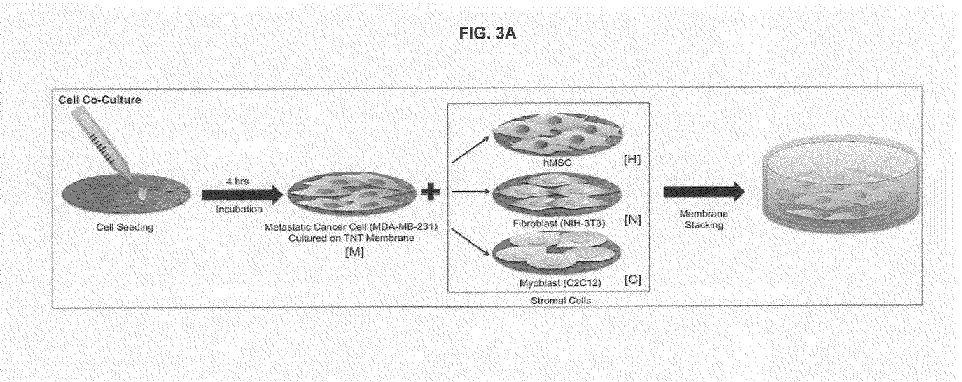

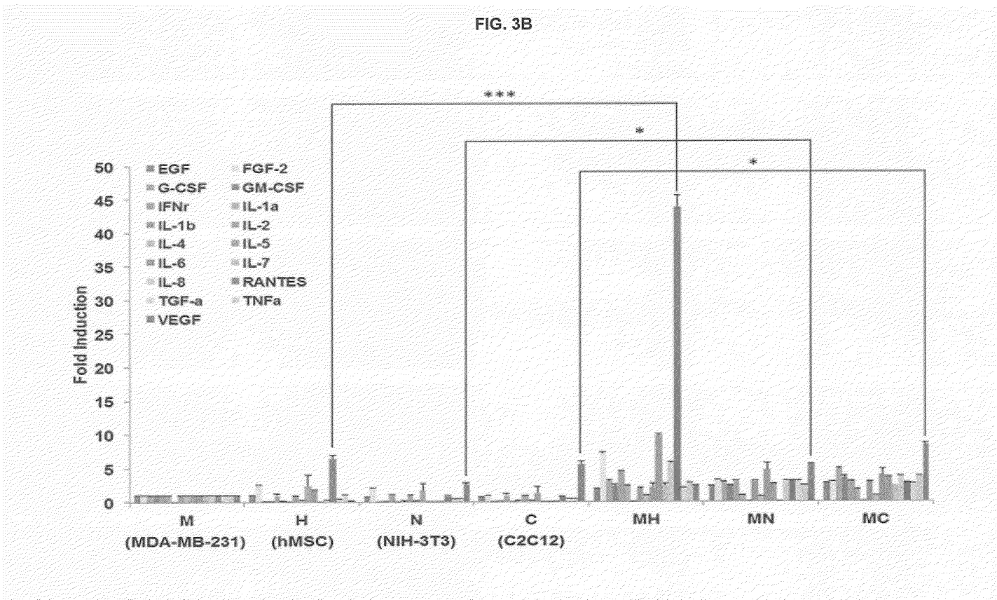

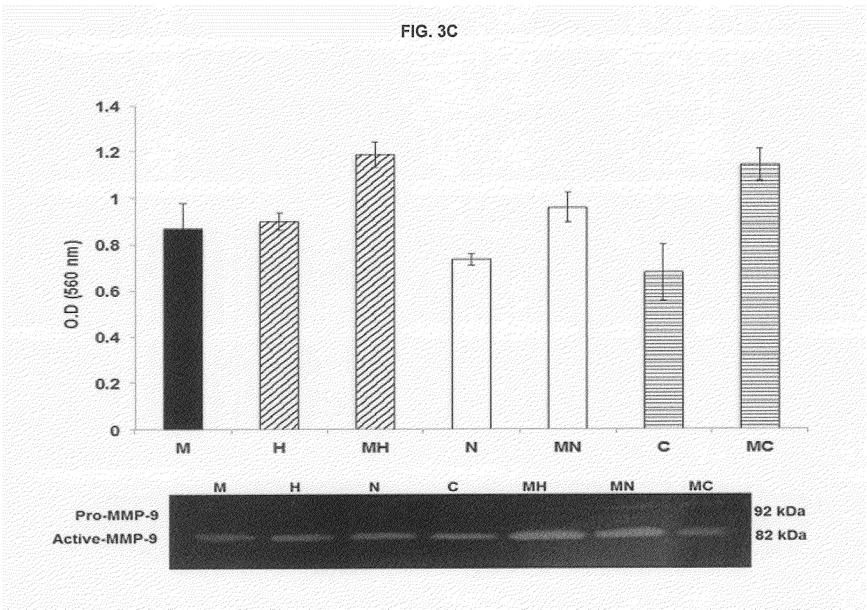

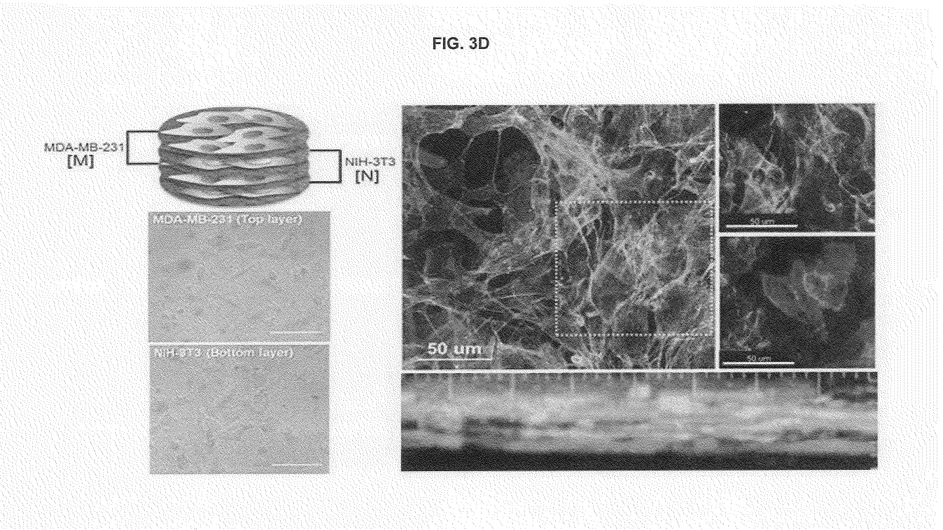

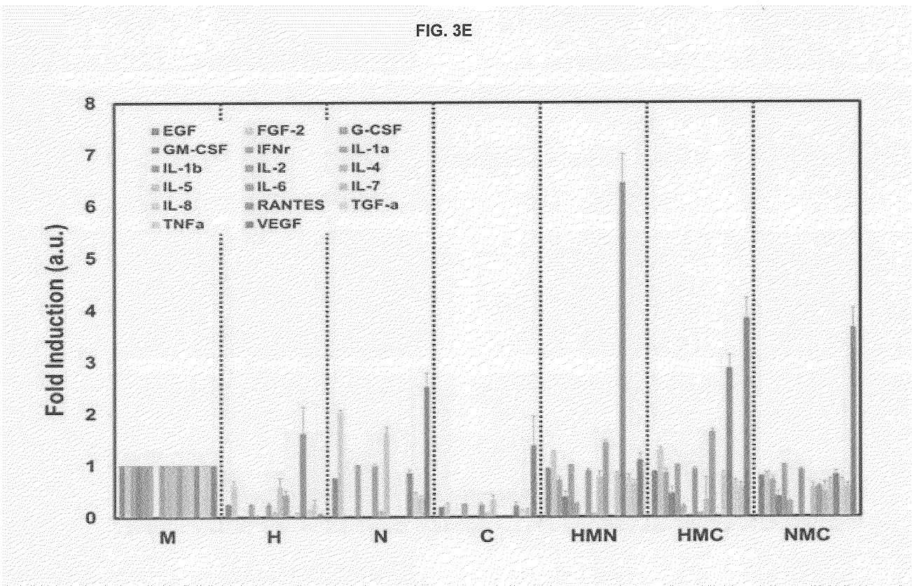

FIG. 3A is a schematic representation of the co-culture of metastatic cancer cells (MDA-MB-231) and three different types of stromal cells using stacking TNT membranes containing each cell line. The stacking for co-culture was performed as follows. Breast cancer cells (Cell Conc.=6.6×10⁴) and stromal cells (Cell Conc.=3.3×10⁴), cell number ratio was 2:1, were seed to transparent, nanoporous and transferable (TNT) cellulose acetate (CA) membrane. We used the hMSC with under 7th passages. To prevent the floating of TNT membrane during cell culture, stainless ring that has a diameter almost the same as TNT membrane press the membrane. After 4 hrs of cell seeding (this time was enough for cell adhesion on the CA film, so could prevent direct contact between metastatic cancer cell and stromal cells), cancer cultured film and stromal cultured film were stacked in a clean well to observe cell-cell interactions. For more exclusive observation of co-culture effect, cells were cultured for 2 days at 37° C., and 5% CO₂ condition.

### Example 3

### Demonstration of Intercellular Communication Through Paracrine Interaction

As shown in FIG. 3A, the cell-cell communication through paracrine was assessed using the metastatic breast cancer cells (MDA-MB-231) and three different types of cells [hMSC (H), fibroblast NIH-3T3 (N), and myoblast C2C12 (C)] using the membrane as prepared in Example 1 as described below. The growing tumor cells actively recruit hMSCs considered as a tumor-associated stromal cell through the secretion of various endocrine and paracrine signal. Fibroblasts and myoblasts also significantly affect the growth and progression of tumor via releasing of cytokine in-vivo (Nicolini, A., Carpi, A. & Rossi, G. Cytokines in breast cancer. Cytokine &amp; Growth Factor Reviews 17, 325-337 (2006)). Accordingly, four different cell lines [metastatic cell line (M) and three stromal cell lines (H, N and C)] were used to investigate the concerted interactions between cancer cells and stromal cells. As shown in FIG. 3B, the results of the cell co-stacking assay using the Luminex bead-based suspension array system showed that there was a significant increase in RANTES levels for the M_{bottom}Hₜₒₚ co-stacking case, while noticeably increased VEGF amount was observed for both the M_{bottom}Nₜₒₚ and M_{bottom}Cₜₒₚ co-stacking cases.

### Example 4

### Cytokine Assay

The cell culture media with different co-cultivated cell lines were analyzed to measure the levels of various cytokines, chemokines and growth factors using a Luminex-based suspension array system. After 24 hrs, the media were collected and analyzed for cytokine levels. After culture of cells, we used Milliplex Map (Merck Millipore, MA, USA) to detect seventeen different cytokines [EGF, FGF-2, G-CSF, GM-CSF, IFNY, RANTES, IL-1α, IL-1β, IL-2, IL-4, IL-5, IL-6, IL-7, IL-8, TGFα, TNFα, and VEGF]. Milliplex Map is based on the luminex that uses proprietary techniques to internally color-code microspheres with two fluorescent dyes. Cells were cocultured as described in Example 2. After culture of cells, Milliplex Map (Merck Millipore, MA, USA) was used according to the manufacturer's instruction to detect seventeen different cytokines [EGF, FGF-2, G-CSF, GM-CSF, IFNY, RANTES, IL-1α, IL-1β, IL-2, IL-4, IL-5, IL-6, IL-7, IL-8, TGFα, TNFα, and VEGF].

Briefly, the sample was incubated with bead which is coated with specific biotinylated-antibody. And then, the reaction mixture was incubated with streptavidin PE conjugate. The signal from microspheres was detected by a laser, which excite internal dyes marking the microsphere set. We repeated six times this experiment for test of reproducibility and error bars. And we obtained the standard curve of each cytokine and background signal from cell culture media for normalization of data.

Results are shown in FIG. 3B. When cells were cultured alone, very high concentration of RANTES was observed in hMSC (H). In NIH-3T3 (N) and C2C12 (C) cells, relatively high level of VEGF (vascular endothelial growth factor) was observed. When cells are co-cultured, significantly increased amount of RANTES was observed in M_{bot}Hₜₒₚ and increased amount of VEGF was observed in M_{bot}Nₜₒₚ and M_{bot}Cₜₒₚ These results suggest that the inductions and changes in the amount of characteristic cytokines from metastatic MDA-MB-231 cells are closely associated with neighboring stromal cell types as well as that the TNT membranes can serve as co-culture platforms allowing for accurate analysis on the cytokine-mediated cell-cell communications

### Example 5

### Analysis of Cell Migration

The migration changes of metastatic cancer cell were assessed using a transwell system (ECM 508, Merck Millipore, MA, USA) and Gelatin Zymography.

For transwell system analysis, first, each cell line was cultured on CA film (Cell Conc.=1.0×10⁵) and cells on three different co-culture conditions (breast cancer cell:stromal cell ratio=2:1, Total cell conc.=1.0×10⁵) were grown for 2 days. After 2 days of cell culture, the media was collected and 500 μL was transferred to the bottom feeder tray of migration assay kit (ECM 508, Merck-Millipore, USA) for observing the effect of cytokine in media on cancer cell migration. MDA-MB-231 cells were cultured at 37° C., 5% CO₂ and propagated in RPMI supplemented with 10% FBS at 100% confluency. These cells were harvest and were resuspended to 1×10⁶ cells per mL. The 300 μL of cells was added per well and was placed onto the top of the insert-well which contains a microporous polycarbonate membrane with 8 μm pore size. The plate was covered and was incubated for 24 hours at 37° C. in a CO₂ incubator (5% CO₂). Migratory cells moved through the pores of the membrane and clung to the bottom of the polycarbonated membrane, in responses to some signaling molecules loaded into the bottom feeder tray. The cells and media from the top side of the insert were removed carefully by pipetting out the remaining cell suspension. And the insert chamber was moved into a clean well containing 400 μL of cell stain solution. After incubation for 20 minutes at room temperature, the chamber was dipped into a beaker of water several times to rinse. After washing, non-migration cells layer was removed through a cotton-tipped swab. And then transfer the stained insert chamber to a clean well containing 200 μL of Extraction buffer for 15 minutes at room temperature. The stain from the underside was extracted by gently tilting the insert back and forth several times during incubation. Finally, extracted solution (100 μL) transferred to 96-well plate for colorimetric measurement at 560 nm.

For gelatin zymography, after culture of cells on co-culture condition or not for 2 days, the media were collected. For zymography assays which could determine the expression of MMPs, all of the conditioned medium were quantified and were diluted by the addition of sample buffer (sample buffer 0.5M contains Tris HCl 0.2 M, SDS 4%, Glycerol 40%, Bromophenol blue 0.004%) that did not contain mercaptoethanol and was not boiled. The prepared samples were loaded into 10% SDS-polyacrylamide gels containing 0.2% gelatin substrate and were separated (Hoefer, MA, USA). After electrophoresis, the gels were washed in 50 mM Tris-HCl (pH 7.4) containing 2.5% Triton X-100. The gels were then washed in 50 mM Tris-HCl (pH 7.4) buffer, followed by incubation in buffer containing 50 mM Tris-HCl (pH 7.4), 0.02% sodium azide and 10 mM CaCl₂. After washes in deionized water, the gels were stained with 0.25% Coomassie Blue, destained in 10% methanol containing 10% acetic acid.

Results are shown in FIG. 3C. In the migration assay, the optical density (O.D.) of co-cultured samples (M_{bot}Hₜₒₚ, M_{bot}Nₜₒₚ and M_{bot}Nₜₒₚ) were higher than single cell line-cultured cases (M, N and C), indicative of the migration of MDA-MB-231 cells is positively affected by the co-culture of stromal cells. Tumor cells also need to cross basement membranes to invade into surrounding tissues as the tumor mass expands in size, which requires a help of MMP (Matrix metalloproteinase) that can degrade the components of basement membrane and plays as a facilitator of tumor cell invasion and metastasis. MMP has been considered as a hall marker of tumor metastasis. As shown in gelatin zymography results, co-activated interactions between MDA-MB-231 cells and stromal cells show the stronger active MMP bands than the single cell line-cultured cases. It is known that RANTES acts in a paracrine fashion on cancer cells to increase their mobility, invasion and metastasis, and the increase of RANTES secretion could also promote the expression of MMP 9 (Azenshtein, E. et al. The CC Chemokine RANTES in Breast Carcinoma Progression. Cancer Research 62, 1093-1102 (2002)). Also, VEGF is an important factor for tumor proliferation and migration by stimulating angiogenesis that is typically secreted from mammary or stromal (myo) fibroblasts (Mishra, P. et al. Chemokines at the crossroads of tumor-fibroblast interactions that promote malignancy. Journal of Leukocyte Biology 89, 31-39). These results suggest that increased VEGF amount with M_{bot}Nₜₒₚ and M_{bot}Cₜₒₚ co-cultures played an important role in activating the migration and invasiveness of the MDA-MB-231 cells. These results also indicate that the TNT membrane-based cell co-stacking assay may be useful for selectively identifying key cytokines or growth factors leading to specific cell phenotypic results via cell-cell communications.

### Example 6

### Actin Staining of the Co-Cultured Cells

After cell co-culture via 4-layer stacking, cell culture medium was harvested, and cells were washed twice with PBS solution. Cells were then fixed by 4% paraformaldehyde/PBS for 15 min. Cells were washed with PBS solution three times (5 min for each time). To block nonspecific binding, we used 1% BSA/PBS/0.3% tween20 for 15 min, and the cells were washed with PBS solution afterwards. And then each layer was separated and was moved to empty well to label cell individually. Phalloidin-Tetramethyl rhodamine isothiocyanate (TRITC) (Sigma-Aldrich, CA, USA) and phalloidin-Fluorescein isothiocyanate (FITC) (Sigma-Aldrich, CA, USA) was diluted to 5% with PBS solution. The Phalloidin-TRITC solution was added to MDA-MB-231 contained well and the Phalloidin-FITC solution was used to stain of three stromal cell lines. After incubation, cells were washed with PBS solution two times. Finally, the stained cell samples were mounted by mounting buffer (Abcam, United Kingdom) and were examined under a confocal laser scanning microscopy (Nikon, Japan).

To check the cross-contamination between cells on bottom layer and cells on top layer during co-culture, we stained live breast cancer cell. Staining reagents (BacMam, Invitrogen, USA) added into plate the cells at the desired density on TNT membrane. GFP-labeled molecule was used to stain the actin of live breast cancer cell. Cells within reagent were incubated for overnight. After incubation, the layer stacked to make cellular multilayer. After culture of cells for 2 days, each layer was separated to obtain the fluorescence images.

Results are shown in FIG. 3D. The transparent characteristic of TNT membrane allows for in-situ monitoring the cell morphology on each stacked-layer by optical microscopy and the confocal laser scanning microscopy (CLSM; Eclipse 90i, Nikon). Also when cells were checked for the cell apoptosis that can be induced by nutrient and oxygen gradient in multicellular co-stacking condition, it was found that every cell line was alive and maintained each proliferation rate without the cross-contamination during the co-culture by TNT membrane multi-stacking up to 4 layers.

Further the cytokine assay shows that TNT-based multicellular co-culture system demonstrated the intercommunications between multi-stacked cells is also maintained, as shown in FIG. 3E. FIG. 3E is the result of the cytokine assay after the co-culture with three different cell lines multi-stacked with TNT membrane, which indicates that cell lines during co-culture with TNT membranes recognize each other by paracrine signaling, and, in this reason, produce the different amount and kinds of cytokines depending on the cell types used in co-culture. Such multi-stacked TNT platforms of the present disclosure thus can be used as system for a simultaneous co-culture of more than one different cells types to observe and to understand the behavior of co-existed cells in an environment similar to that of in vivo.

### Example 7

### TNT Membrane-Based Cell Transfer and Shuffling (TNT-CTS) Assay

The shuffling assay by translocation and reassembly of TNT membrane was designed for screening the cytokines, which were released specifically by intercommunication between MDA-MB-231 and three stromal cells. First, cells were seeded onto the TNT membrane which was then stacked in the following combination [MDA-MB-231 with hMSC/MDA-MB-231 with NIH-3T3/MDA-MB-231 with C2C12]. After 24 hrs of cell culture, the media were collected to analysis the cytokines, and the TNT membrane of MDA-MB-231 co-cultured with one type of stromal cell was translocated to the TNT membranes containing other type of stromal cells as follows: [hMSC→NIH-3T3, C2C12/NIH-3T3→hMSC, C2C12/C2C12→hMSC, NH-3T3].

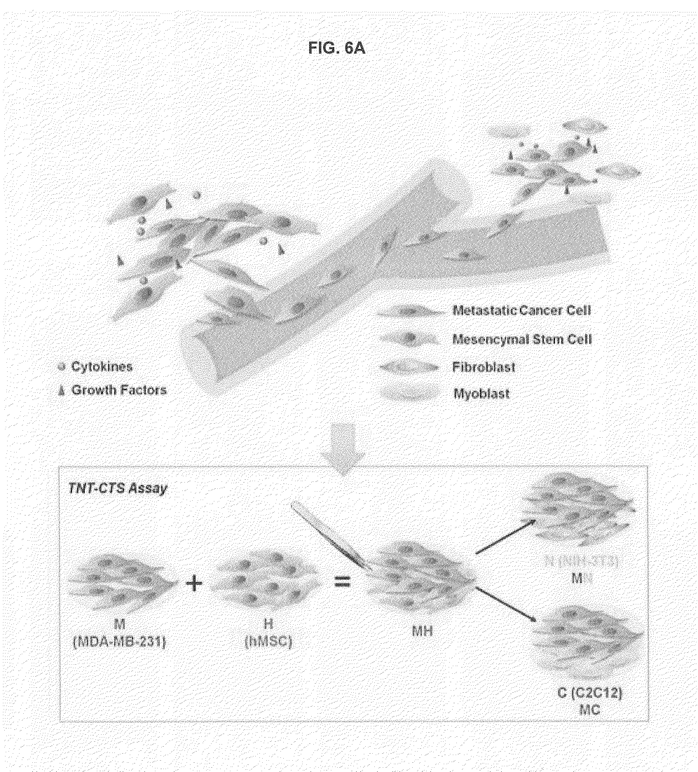

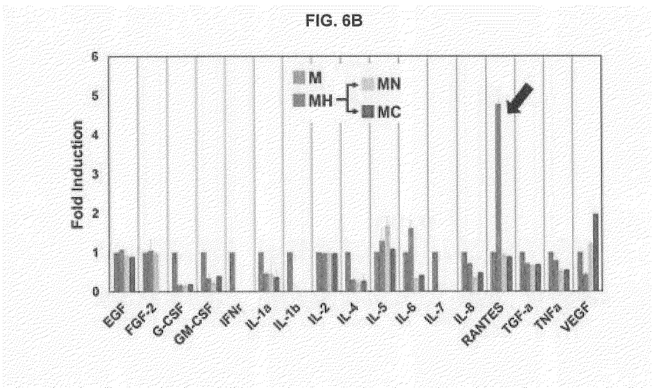

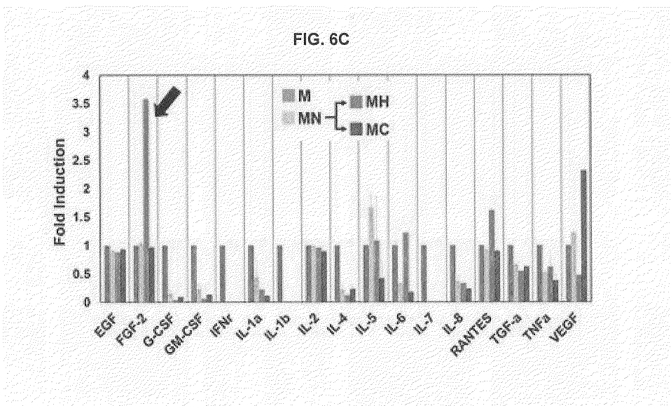

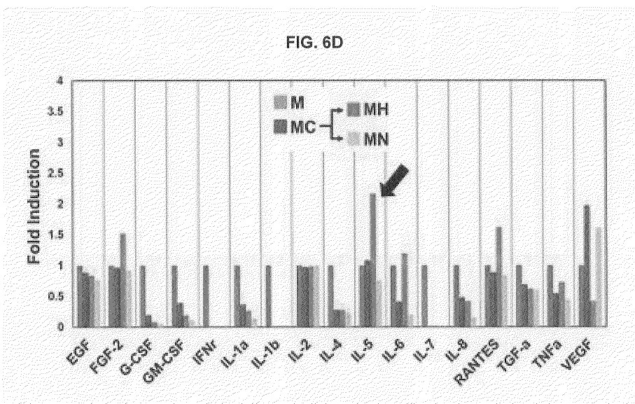

Results are shown in FIG. 6A to 6D. On the basis of the transferability of TNT membranes, we designed and performed the TNT membrane-based cell transfer and shuffling (TNT-CTS) assay, which enabled us to trace the history of sequential stromal activations and to control the sequence of stromal cell co-culture with metastatic cancer cells. The sequential co-culture of metastatic cancer cells with different types of stromal cells offers systematic ways of understanding of cancer progress by mimicking and simplifying complex in vivo tumor microenvironment via controllable sequential signaling between metastatic cells and different types of stromal cells during metastasis (FIG. 6A). The different expressions of various cytokines and growth factors were detected when the same stromal cell lines were used with different co-culture sequences. In particular, the major protein species and amount, produced from MH, largely varied depending on the culture sequences. RANTES was the major protein when there was no pre-culture (FIG. 6B), but FGF-2 and IL-5 were the major proteins when M was pre-cultured with N and C, respectively (FIGS. 6C and 6D).

The present TNT membrane sequential co-culture (shuffling) can be advantageously used not only for autocrine signaling in activated stromal cells, but also for efficient tool for study the paracrine interaction-mediated sequential activation of many different types of stromal cells during cancer metastasis. Further it was clearly confirmed that the different expression of cytokines is detected according to the designed sequence of co-cultured stromal cells, even though the combination of co-cultured cell line is same. For example, the release of FGF-2 was increased in MH under the sequence effect of MN interaction whereas IL-5 was stimulated in same MH condition under the influence of MC communications. Notably, the level of specific cytokines, FGF2 and IL-5, on each condition reflected synergistic interactions between stromal cells in sequential co-culture, as they are higher than those produced by M culture alone.

The present TNT membrane shuffling co-culture method can be used advantageously for a study of cancer development and finding new target molecules for cancer treatment through screening the signaling molecules. Further the present system can be applied in the field of control the differentiation of stem cells by changing the sequence of surrounding environmental factors including other cell types.

The TNT membrane-based co-culture platform facilitates the detailed analysis of the intercommunication between metastatic cancer cell and several types of stromal cells based on the advantages of facile stacking and destacking originated from the transparency, transferability and nanoporous structures. The present system can also advantageously used for a straightforward and cost-effective method for cell-co-culture and analysis.

### Example 8

### Demonstration of Intercellular Communication Through Gap Junction Between Breast Cancer Cells and hMSC

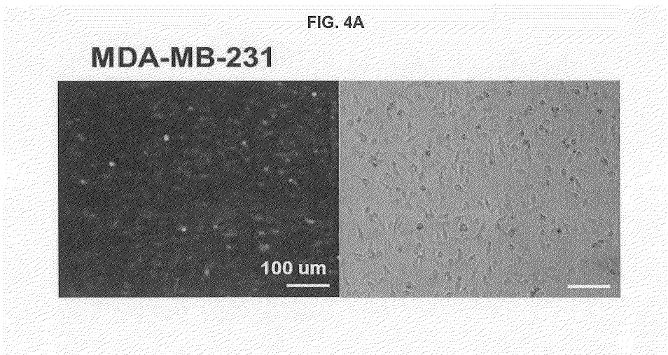

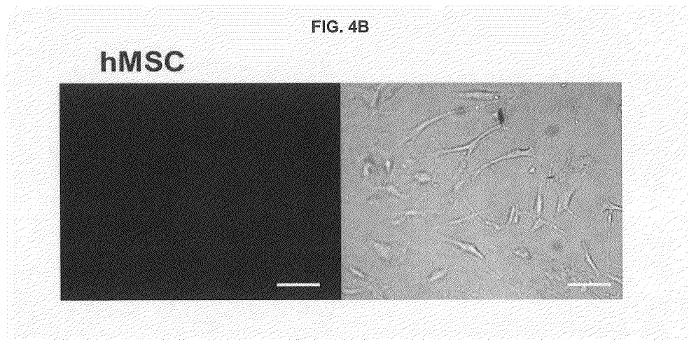

The metastatic breast cancer cells (MDA-MB-231) were labeled with calcein-AM (2.5 μM, Sigma-Aldrich, CA, USA), which can spread only through the gap junction. This labeled breast cancer cells were cocultured with unlabeled hMSCs on TNT membranes. After 2 days of cell culture, calcein-AM transfer was evaluated by fluorescence microscope (Carl Zeiss, Germany; 10× objective lens). Results are shown in FIGS. 4A and 4B. The calcein-AM is only transferable through gap junction, which can be formed by direct contact between cells. The data of FIGS. 4A and 4B show that there is no physical contact between MDA-MB-231 and hMSC since Calcein AM can only be transported through the gap junction in cellular membrane into live cells.

### Example 9

### Quantification of Diffused Epidermal Growth Factors (EGF) Through Membrane

The EGF diffusion through nanoporous membrane was studied as increase the thickness of membranes increases. To control the thickness of membrane, the single TNT membrane was stacked in 3 layers, 5 layers, and 8 layers. The single TNT membrane and stacked TNT membranes were placed between wells of a chamber. Membranes separated the wells. We added EGF proteins-contained PBS solution into one well and added same volume of PBS solution into the other well. The protein diffusion was induced by a concentration difference between the solutions on both sides of the membranes. After 2 days incubation at 37° C., we quantified the amount of diffused proteins using Bradford assay (Bio-Rad, CA, USA).

Further to verify the diffusion of protein through TNT membrane, we fabricated chamber. TNT membrane located between chambers which contained 80 μg/ml of EGF (E9644, Sigma Aldrich, St. Louis, Mo., USA) in PBS with 10% FBS (EGF chamber) and PSB with 10% FBS (buffer chamber), respectively. The EGF diffused from EGF chamber to buffer chamber. As incubation time increased, we collected solution on side of buffer chamber (time point; 1, 3, 6, 12, and 24 h). To quantify the amount of EGF in buffer solution at different time point, we performed sandwich ELISA. 96 well was coated with rabbit-polyclonal antibody to EGF (ab9695, Abcam, Cambridge, England) in carbonate/bicarbonate buffer (pH 9.7). After blocking the remaining protein-binding sites in the coated wells by adding 200 μl blocking buffer, 5% non fat dry milk/PBS, we added samples to each well. To accurate quantify the amount of EGF, we also added standard samples (0, 1, 5, 10, 50 and 100 μg/ml). After 90 min incubation and three times washing, we added mouse-monoclonal antibody to EGF (ab10409, Abcam, Cambridge, England) to each well. After washing the plate four times with TBST buffer, anti-mouse antibody-horse radish peroxidase (HRP), 2nd antibody, added and incubated for 1 h. To obtain the signal, we added substrate to HRP; TMB (3,3′,5,5′-tetramethylbenzidine) (N301, Waltham, Mass., USA) solution to each well and incubated for 15-30 min. Finally, we added equal volume of stopping solution (2 M H2SO4) and read the optical density at 450 nm.

Results are shown in FIG. 5A to 5D. When the membrane thickness effect on the paracrine signaling between cells was checked using the 10-μm-thick 20 wt % CA membrane and the 480-nm thick 4 wt % CA membrane and the PET membrane, the results indicated that the 480-nm-thick 4 wt % CA membrane, prepared at 65% RH, yielded the largest RANTES expression from the cells (FIG. 5A). The test studying the effect of the distance between cells on cytokine secretion by increasing the number of the stacking showed that the amount of secreted RANTES as the result of cell-cell paracrine communications was strongly correlated with the cell-cell distance (FIG. 5B). In the paracrine signaling, the signaling molecules released by a cell affect target cells in close proximity because they diffuse away from the signaling cell, forming a concentration gradient and also inducing different cellular responses depending on the distance of a target cell from the signal-releasing site. In this regard, the close proximity between co-cultured cells is a major parameter in cell-cell signaling, and the present TNT membrane can offers such an environment by keeping the sub-μm intercellular distance between different cell lines. Furthermore, when the diffusion of epidermal growth factors (EGFs) through the TNT membrane with a two chamber system with one chamber filled only with buffer solution and the other chamber filled with EGFs in buffer solution was checked, (FIGS. 5C and 5D). It was showed that the concentration gradient of EGFs across the membrane was clearly observed, and the amount of diffusing EGF decreased as the number of the stacking layers was increased (FIGS. 5C and 5D). Importantly, the result from the sandwich-ELISA for EGF that diffuses into the buffer chamber shows that the saturation of EGF in the buffer chamber occurs after 6-h incubation in PBS with 5% FBS, and the protein concentration remains the same even after 24-h incubation. The data support that the present TNT membrane with porous structure and sub-μm thickness allows the efficient protein transfer between cells that reside in different compartments and also shows that the first 6 hour is critical for the protein diffusion through the membrane pores.

While the present invention has been shown and described in terms of various aspects, it will be apparent to those skilled in the art that various modification and changes may be made without departing the principles and spirit of the invention. Thus the scope of the invention must be defined by the appended claims and their equivalents.

With respect to the use of substantially any plural and/or singular terms herein, those having skill in the art can translate from the plural to the singular and/or form the singular to the plural as is appropriate to the context and/or application. The various singular/plural permutations may be expressly set forth herein for sake of clarity.

1. A method for preparing a transferable membrane having a nanometer scale dimension in thickness and pore size by non-solvent vapor-induced phase separation process, the method comprising spin-casting a polymer solution in a closed humid chamber and controlling the relative humidity (RH) of the chamber using at least one supersaturated salts solution whereby the size of the pores is controlled.

2. The method of claim 1, wherein the polymer is cellulose acetate, polysulfone, polyethersulfone, polyarylonitrile, cellulosics, poly(vinylidene fluoride), poly(tetrafluoroethylene), polyimide or polyamide.

3. The method of claim 1, wherein the at least one supersaturated salt is LiCl, CaCl₂, MgCl₂, KCO₃, NaBr, NaCl, or KCl.

4. The method of claim 1, wherein the at least one supersaturated salt is CaCl₂ and KCl and the relative humidity is increased from 25 to 85% wherein the CaCl2 is used when the RH is 25-45% and KCl is used when the RH is 55-85%.

5. The method of claim 4, wherein the relative humidity is gradually increased from 25 to 85% or increased in a stepwise manner from 25, 35, 45, 55, 65, 75 and 85%.

6. The method of claim 1, wherein the relative humidity is controlled at RT or 30° C.

7. The method of claim 1, wherein the thickness of the membrane is controlled by a spinning rate, a polymer concentration and/or the solubility of the polymer in a solvent.

8. A cell culture platform comprising: at least one membrane prepared according to claim 1 having a plurality of pores in nanoscale extending between opposite sides thereof and a thickness of about 500 nm or less; and a first and a second chamber separated by the membrane.

9. The cell culture platform of claim 8, wherein the first and second chambers are able to communicate with each other through the plurality of pores in nanoscale.

10. The cell culture platform of claim 8, wherein the first and second chambers contain a cell culture medium, the first chamber contains a first cell type, and the second chamber contains a second cell type.

11. The cell culture platform of claim 10, wherein the first cell type and the second cell type are identical or different, or at least one of the first cell type and the second cell type is a mixture of at least two different cell types.

12. The cell culture platform of claim 8, which further comprises at least one intermediate chamber positioned between the first chamber and the second chamber, the intermediate chamber being separated from each of the first and second chambers by at least one nanoscale membrane.

13. The cell culture platform of claim 12, wherein the intermediate chambers is comprised in 2 to 10.

14. The cell culture platform of claim 12, wherein the first, second and intermediate chambers contain a cell culture medium, and at least one of the chambers contain a same or a different cell type or a mixture of at least two different cell types.

15. The cell culture platform of claim 8, wherein the density of the pores of the nanoscale membrane is 10⁵ to 10⁶ pores per cm².

16. The cell culture platform of claim 8, wherein the average diameter of the pores of the nanoscale membrane is 50-100 nm.

17. A method for co-culturing two or more types of cells, comprising:
providing at least one membrane according to claim 1, in which when the membranes comprised are two or more, the membranes are stacked in layers from top to bottom or from right to left; seeding cells on at least one of the membranes wherein the cells are seeded on either side of each membrane and the cells seeded are of the same or different type or a mixture of at least two different types of cells; and
culturing the cells whereby the cells are able to communicate with each other through the pores of the membrane.

18. The method of claim 17, wherein the communication is by a direct contact including a gap junction or a tight junction or by an indirect contact via exchange of soluble factors through paracrine or endocrine signaling.

19. The method of claim 17, wherein the cells seeded are cancer cells or stromal cells, and each of the cancer cells and stromal cells are seed on a different membrane.

20. The method of claim 17, wherein the positions of the membranes are changed relative to each other, or at least one of the membranes is removed and replaced with a fresh membrane, and the fresh membrane contains cells that are different from the cells in the existing membranes.

21. A cell co-culture kit comprising: the membrane prepared according to the methods of claim 1 or comprising the platform of claim 7; one or more cell culture media; one or more cell lines; and instructions for co-culturing the one or more cell lines using the kit.
